(19) United States
(12) Patent Application Publication
(10) Pub. No.: US 2008/0227792 A1
(43) Pub. Date: Sep. 18, 2008

(54) 3,4-Dihydro-1H-Isoquinoline-2-Carboxylic Acid 5-Aminopyridin-2-Yl Esters

(75) Inventors: Johannes Cornelis De Jong, Bagsvaerd (DK); Poul Jacobsen, Slangerup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/816,299

(22) PCT Filed: Feb. 13, 2006
(86) PCT No.: PCT/EP2006/050882

§ 371 Date: Apr. 18, 2008

(30) Foreign Application Priority Data
Feb. 15, 2005 (DK) PA 2005 00222
Feb. 28, 2005 (DK) PA 2005 00295

(51) Int. Cl. A61K 31/496; A61K 31/4725; C07D 401/14; A61P 3/04; C07D 403/14

(52) U.S. Cl. 514/253.05; 546/147; 514/307; 514/278; 544/363

(57) ABSTRACT

Novel compounds of formula (I), pharmaceutical compositions comprising them and use thereof in the treatment and/or prevention of diseases and disorders related to hormone sensitive lipase. More particularly, the compounds are useful for the treatment and/or prevention of diseases and disorders in which modulation of the activity of hormone sensitive lipase is beneficial.

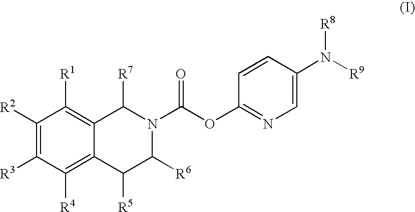

### FIELD OF THIS INVENTION

This invention relates to the novel compounds mentioned in claim 1, below, to pharmaceutical compositions comprising these compounds, to the use of these compounds as pharmaceutical compositions, and to methods of treatment employing these compounds and compositions. The compounds of formula I show strong inhibition of hormone sensitive lipase. As a result, the compounds are useful for the treatment and/or prevention of diseases and disorders related to hormone sensitive lipase.

### BACKGROUND OF THIS INVENTION

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed in adipose tissue, macrophages, muscle, adrenal, testis and islets (Kraemer and Shen, J. Lipid Res. 2002, 43, 1585-1594). In the adipocytes, HSL catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is primarily mediated through the cAMP-protein kinase A and AMP-dependent kinase pathways. There are compounds like nicotinic acid and its derivatives, that decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, since these pathways are used by other processes in the body, these drugs have severe side effects.

The object of this invention is to overcome or ameliorate at least one of the disadvantages of the prior art, or to provide a useful alternative, for example:
I) to provide compounds and pharmaceutical compositions that inhibit the lipolytic activity of HSL or
II) to provide compounds which have good pharmaceutical properties such as solubility, bioavailability, specificity etc.

Definitions

The term “halogen” in the present context designates an atom selected from the group consisting of F, Cl, Br and I.

The term “C₁₋₆-alkyl” in the present context designates a saturated, branched or straight hydro-carbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₂₋₆-alkyl” in the present context designates a saturated, branched or straight hydro-carbon group having from 2 to 6 carbon atoms. Representative examples include, but are not limited to, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆-alkoxy” in the present context designates a group of the formula —O—C₁₋₆-alkyl wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₃₋₆-alkoxy” in the present context designates a group of the formula —O—C₃₋₆-alkyl wherein C₃₋₆-alkyl is a saturated, branched or straight hydrocarbon group having from 3 to 6 carbon atoms. Representative examples of C₃₋₆-alkoxy include, but are not limited to, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₂₋₆-alkenyl” as used herein, represent an olefinically unsaturated branched or straight hydrocarbon group having from 2 to 6 carbon atoms and at least one double bond. Examples of such groups include, but are not limited to, vinyl, 1-propenyl, 2-propenyl, allyl, isopropenyl, 1,3-butadienyl, 1-butenyl, hexenyl, pentenyl and the like. In said alkenyl moiety, the two “free” bonds may be connected to the same atom (often designated spiro compounds) or they may be connected to two different atoms.

The term a “free bond” as used herein represents the positions where the group in question is connected to another group.

The term “C₃₋₁₃-cycloalkyl” as used herein represents a saturated mono-, bi-, tri- or spiro-carbocyclic group having 3 to 13 carbon atoms, preferably from 3 to 10 carbon atoms. Representative examples are cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “heterocyclyl” as used herein represents a saturated 3 to 13 membered monocyclic ring, containing one or more heteroatoms selected from nitrogen, oxygen, sulfur, —S(═O)— and —S(═O)₂—Representative examples are aziridinyl (for example, aziridin-1-yl), azetidinyl (for example, azetidin-1-yl and azetidin-3-yl), oxetanyl, pyrrolidinyl (for example, pyrrolidin-1-yl, pyrrolidin-2-yl and pyrrolidin-3-yl), imidazolidinyl (for example, imidazolidin-1-yl, imidazolidin-2-yl and imidazolidin-4-yl), oxazolidinyl (for example, oxazolidin-2-yl, oxazolidin-3-yl and oxazolidin-4-yl), thiazolidinyl (for example, thiazolidin-2-yl, thiazolidin-3-yl and thiazolidin-4-yl), isothiazolidinyl, piperidinyl (for example, piperidin-1-yl, piperidin-2-yl, piperidin-3-yl and piperidin-4-yl), homopiperidinyl (for example, homopiperidin-1-yl, homopiperidin-2-yl, homopiperidin-3-yl and homopiperidin-4-yl), piperazinyl (for example, piperazin-1-yl and piperazin-2-yl), morpholinyl (for example, morpholin-2-yl, morpholin-3-yl and morpholin-4-yl), thiomorpholinyl (for example, thiomorpholin-2-yl, thiomorpholin-3-yl and thiomorpholin-4-yl), 1-oxothio-morpholinyl, 1,1-dioxo-thiomorpholinyl, tetrahydrofuranyl (for example, tetrahydrofuran-2-yl and tetra- hydrofuran-3-yl), tetrahydrothienyl, tetrahydro-1,1-dioxothienyl, tetrahydropyranyl (for example, 2-tetra-hydropyranyl), tetrahydrothiopyranyl (for example, 2-tetrahydrothiopyranyl), 1,4-dioxanyl, 1,3-dioxanyl, and the like. Heterocyclyl is also intended to represent a saturated 6 to 13 membered bicyclic ring containing one or more heteroatoms selected from nitrogen, oxygen, sulfur, —S(═O)— and —S(═O)₂—. Representative examples are octahydroindolyl (for example, octahydroindol-1-yl, octahydroindol-2-yl, octahydroindol-3-yl and octahydroindol-5-yl), decahydroquinolinyl (for example, decahydroquinolin-1-yl, decahydroquinolin-2-yl, decahydroquinolin-3-yl, decahydroquinolin-4-yl and decahydroquinolin-6-yl), decahydroquinoxalinyl (for example, decahydroquinoxalin-1-yl, decahydroquinoxalin-2-yl and decahydroquinoxalin-6-yl) and the like. Heterocyclyl is also intended to represent a saturated 6 to 13 membered ring containing one or more heteroatoms selected from nitrogen, oxygen, sulfur, —S(═O)—and —S(═O)₂— and having one or two bridges. Representative examples are 3-azabicyclo[3.2.2]nonyl, 2-azabicyclo[2.2.1]heptyl, 3-azabicycle[3.1.0]hexyl, 2,5-diazabicyclo[2.2.1]heptyl, atropinyl, tropinyl, quinuclidinyl, 1,4-diazabicyclo[2.2.2]octanyl, and the like. Heterocyclyl is also intended to represent a 6 to 13 membered saturated ring containing one or more heteroatoms selected from nitrogen, oxygen, sulfur, —S(═O)— and —S(═O)₂— and containing one or more spiro atoms. Representative examples are 1,4-dioxaspiro[4.5]decanyl (for example, 1,4-dioxaspiro[4.5]decan-2-yl and 1,4-dioxaspiro[4.5]decan-7-yl), 1,4-dioxa-8-azaspiro[4.5]decanyl (for example, 1,4-dioxa-8-azaspiro[4.5]decan-2-yl and 1,4-dioxa-8-azaspiro[4.5]decan-8-yl), 8-azaspiro[4.5]decanyl (for example, 8-azaspiro[4.5]decan-1-yl and 8-aza-spiro[4.5]decan-8-yl), 2-azaspiro[5.5]undecanyl (for example, 2-azaspiro[5.5]undecan-2-yl), 2,8-diaza-spiro[4.5]decanyl (for example, 2,8-diazaspiro[4.5]decan-2-yl and 2,8-diazaspiro[4.5]decan-8-yl), 2,8-diazaspiro[5.5]undecanyl (for example, 2,8-diazaspiro[5.5]undecan-2-yl), 1,3,8-triazaspiro[4.5]decanyl (for example, 1 ,3,8-triazaspiro[4.5]decan-1-yl, 1 ,3,8-triazaspiro[4.5]decan-3-yl and 1 ,3,8-triazaspiro-[4.5]decan-8-yl), and the like.

The term “aryl” as used herein represents a carbocyclic aromatic ring system being either monocyclic, bicyclic, or polycyclic, such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-di-hydronaphthyl and the like.

The term “aryloxy” as used herein represents an aryl which is linked via an oxygen atom, for example, phenoxy, 1-naphthyloxy, 2-naphthyloxy and the like.

The term “heteroaryl” as used herein represents a heterocyclic aromatic ring system containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadi-azolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxa-zolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, 3,4-dihydroiso-quinolinyl, pyrrolinyl, pyrazolinyl, indolinyl, oxazolidinyl, oxazolinyl, oxazepinyl and the like.

The term “halo-C₁₋₄-alkyl” as used herein refers to C₁₋₄-alkyl, substituted one or more times at any carbon atom(s) with any halogen. Representative examples are trifluoromethyl, 2,2,2-trifluoroethyl, and the like.

The term “halo-C₁₋₄-alkoxy” as used herein refers to C₁₋₄-alkoxy, substituted one or more times at any carbon atom(s) with any halogen. Representative examples are trifluoromethoxy and 2,2,2-trifluoroethoxy, and the like.

The term “ring system” as used herein includes aromatic as well as non-aromatic ring moieties, which may be monocyclic, bicyclic or polycyclic, and they encompass moieties with zero, one or more heteroatoms selected from nitrogen, oxygen and sulfur. Non-limiting examples of such ring systems are aryl, C₃₋₈-heterocyclyl and heteroaryl.

The term “heterocyclic system” as used herein includes aromatic as well as non-aromatic ring moieties, which may be monocyclic, bicyclic or polycyclic, and containing in their ring structure one or more heteroatoms selected from nitrogen, oxygen and sulfur. Non-limiting examples of such hetero-cyclic systems are C₃₋₈-heterocyclyl and heteroaryl.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent, the substituents may be the same or different.

The term “optionally covalently bound” as used herein means that the substituents in question are either not covalently bound to each other or the substituents are directly connected to each other by a covalent bond. A non-limiting example of such optionally covalently bound substituents is -N-ethyl-n-propyl which provided that the substituents, ethyl and n-propyl, are optionally covalently bound may be -N-ethyl-n-propyl, 1-piperidyl, 3-methyl-1-pyrrolidyl or 2,3-dimethyl-1-azeti-dyl.

The term “oxo” shall mean the radical ═O (the bonds being connected to the same atom).

The term “thioxo” shall mean the radical ═S (the bonds being connected to the same atom).

The group —S(═O)₂(OH) may also be designated sulfo.

Mercapto may also be designated sulfanyl.

The terms “disease”, “condition” and “disorder” as used herein are used interchangeably to specify a state of a patient which is not the normal physiological state of man.

The term “treatment” as used herein means the management and care of a patient having developed a disease, condition or disorder, as well as the management and care of an individual at risk of developing the disease, condition or disorder prior to the clinical onset of said disease, condition or disorder. The purpose of treatment is to combat the disease, condition or disorder, as well as to combat the development of the disease, condition or disorder. Treatment includes the administration of the active compounds to prevent or delay the onset of the symptoms or complications and to eliminate or control the disease, condition or disorder as well as to alleviate the symptoms or complications associated with the disease, condition or disorder.

The term “effective amount” as used herein means a dosage which is sufficient in order for the treatment of the patient to be effective compared with no treatment.

The term “modulate” as used herein means to influence, i.e. to modulate a parameter means to influence that parameter in a desired way. Examples are to modulate insulin secretion from beta cells and to modulate the plasma level of free fatty acids.

The term “medicament” as used herein means a pharmaceutical composition suitable for administration of the pharmaceutically active compound to a patient.

The term “pharmaceutically acceptable” as used herein means suited for normal pharmaceutical applications, i.e. giving rise to no adverse events in patients etc.

### DESCRIPTION OF THIS INVENTION

In one aspect, this invention relates to the compounds of formula I defined in claim 1 below.

Specific embodiments, aspects and features of this invention are illustrated in the following embodiments a) et seq.:

a) Compounds of formula I as defined in claim 1 below.

b) Compounds of formula I according to embodiment a), wherein R¹ is hydrogen.

c) Compounds of formula I according to any one of the preceding embodiments, wherein R² is hydrogen, alkoxy or halogen, preferably hydrogen, bromo, chloro, fluoro or methoxy.

d) Compounds of formula I according to the preceding embodiment, wherein R² is hydrogen or alkoxy, preferably methoxy.

e) Compounds of formula I according to any one of the preceding embodiments, wherein R³ is hydrogen, halogen or alkoxy, preferably hydrogen, chloro, fluoro or methoxy.
f) Compounds of formula I according to any one of the preceding embodiments, wherein R³ is hydrogen or alkoxy, preferably methoxy.
g) Compounds of formula I according to any one of the preceding embodiments, wherein R⁴ is hydrogen.
h) Compounds of formula I according to any one of the preceding embodiments, wherein R⁵ is hydrogen.

i) Compounds of formula I according to any one of the preceding embodiments, wherein R⁶ is hydrogen.

j) Compounds of formula I according to any one of the preceding embodiments, wherein R⁷ is hydrogen.

k) Compounds of formula I according to any one of the preceding embodiments, wherein R⁸ is hydrogen.

l) Compounds of formula I according to any one of the preceding embodiments, wherein R⁸ is hydrogen, and R⁹ is 4,5-dihydrothiazolyl substituted with one or two alkoxy groups in the thiazole ring, preferably 4,4-dimethyl-4,5-dihydrothiazol-2-yl; 4,4-diethyl-4,5-dihydrothiazol-2-yl or 4-ethyl-4-methyl-4,5-dihydrothiazol-2-yl.

m) Compounds of formula I according to any one of the preceding embodiments, wherein R⁹ is C₃₋₈-heterocyclyl, optionally substituted by C₃₋₁-cycloalkyl.

n) Compounds of formula I according to any one of the preceding embodiments, wherein R⁹ is 3-thia-1-azaspiro[4.4]non-1-en-2-yl.

o) Compounds of formula I according to any one of the preceding embodiments to the extend possible, wherein R⁸ together with R⁹ and together with the adjacent nitrogen atom is C₃₋₈-hetero-cyclyl which, optionally, is substituted with oxo, with C₁₋₆-alkyl, preferably, methyl, and/or with C₃₋₁₃-cycloalkyl.

p) Compounds of formula I according to any one of the preceding embodiments to the extend possible, wherein R⁸ together with R⁹ and together with the adjacent nitrogen atom is piperidino (1-piperidyl) or piperazinyl, for example, 1-piperazinyl, each of which is optionally substituted with one of more of the following groups oxo and alkyl, where two alkyl substituents in the same position in the piperidino or piperazinyl ring may together form a ring (making it a spiro compound).

q) Compounds of formula I according to any one of the preceding embodiments to the extend possible, wherein R⁸ together with R⁹ and together with the adjacent nitrogen atom is 4,4-dimethyl-2,6-dioxo-3,4,5,6-tetrahydro-2H-pyridinyl; 2,4-dioxo-3-aza-spiro[5.5]undec-3-yl; 4,4-diethyl-2,6-di-oxo-3,4,5,6-tetrahydro-2H-pyridinyl; 4-ethyl-4-methyl-2,6-dioxo-3,4,5,6-tetrahydro-2H-pyridinyl; 7,9-dioxo-8-aza-spiro[4.5]dec-8-yl; 4,4-dimethyl-2,6-dioxo-3,4,5,6-tetrahydro-2H-pyridinyl; 4-ethyl-2,6-dioxopiperazin-1-yl; 4-ethyl-2,6-dioxopiperazi n-1-yl; 4-isobutyl-2,6-dioxopiperazi n-1-yl; 4,4-dimethyl-2-oxo-3,4,5, 6-tetrahydro-2H-pyridi nyl; 4-isopropyl-2,6-dioxo-3,4,5,6-tetrahydro-2H-pyridinyl; or 3,3-dimethyl-2,6-dioxo-3,4,5,6-tetrahydro-2H-pyridinyl.

r) Compounds of formula I according to any one of the preceding embodiments to the extend possible, wherein R⁸ together with R⁹ and together with the adjacent nitrogen atom is 4,4-dimethyl-2,6-dioxo-3,4,5,6-tetrahydro-2H-pyridinyl or 7,9-dioxo-8-azaspi ro[4.5]dec-8-yl.

Obviously, when R⁸ together with R⁹ and together with the adjacent nitrogen atom represents C₃₋₈-heterocyclyl, the above definition for C₃₈-heterocyclyl applies with the proviso that said group has a nitrogen atom in the position in question.

The compounds of formula I can be prepared by methods known per se or analogously with known methods. For example, reference can be made to the following publications concerning processes of making carbamoyl chlorides: Using triphosgene, pyridine in toluene, reference can be made to: Yasuo Koga, Yoshito Kihara, Minoru Okada, Yoshihiro Inoue, Shirou Tochizawa, Kazuyuki Toga, Kazue Tachibana, Yukio Kimura, Takao Nishi and Hiroyoshi Hidaka, Bioorg. Med. Chem. Lett. 1998, 8 (12), 1471-1476. Using phosgene, triethylamine in tetrahydrofuran, reference can be made to: Pingsheng Zhang and Robert E. Gawley, Tetrahedron Lett. 1992, 33 (21), 2945-2948. See also; Laurent Lemoucheux, Jacques Rouden, Meziane Ibazizene, Franck Sobrio, and Marie-Claire Lasne, J. Org. Chem. 2003, 68 (19), 7289-7297.

In another aspect, this invention relates to a pharmaceutical composition comprising a compound of formula I, more precisely a compound according to any one of the above specific embodiments of compounds of this invention, or a pharmaceutically acceptable salt thereof together with a pharmaceutically acceptable carrier or diluent.

Further specific embodiments, aspects and features of this invention are the following embodiments i) et seq.:

i) A pharmaceutical composition as described herein in unit dosage form, comprising from about 0.05 to about 2000 mg, preferably from about 0.1 to about 500 mg and even more preferable from about 1.0 to about 100 mg of said compound according to this invention or pharmaceutically acceptable salt thereof.

ii) A pharmaceutical composition as described herein for use as a medicament for inhibiting the lipolytic activity of hormone-sensitive lipase against triacylglycerols, diacylglycerols, cholesterol acyl esters or steroid acyl esters, said composition comprising a compound according to this invention or a pharmaceutically acceptable salt thereof together with a pharmaceutically acceptable carrier or diluent.

iii) A pharmaceutical composition as described herein which is for oral administration.

iv) A pharmaceutical composition as described herein which is for nasal, transdermal, pulmonal, or parenteral administration.

v) The use of a compound according to this invention for the preparation of a pharmaceutical composition.

vi) Use of a compound according to this invention for inhibition of hormone sensitive lipase.

vii) The use of a compound according to this invention for preparation of a pharmaceutical composition for inhibition of the lipolytic activity of hormone-sensitive lipase against triacylglycerols, diacylglycerols, cholesterol acyl esters or steroid acyl esters.

viii) The use of a compound according to this invention for the preparation of a pharmaceutical composition for the treatment or prevention of any disorder where it is desirable to a) modulate the plasma level of free fatty acids, glycerol, LDL-cholesterol, HDL-cholesterol, insulin and/or glucose; and/or b) modulate intracellular triacylglycerol and cholesterol ester stores, intracellular level of fatty acids, fatty acid esters such as diacylglycerols, phosphatidic acids, long chain acyl-CoA's as well as citrate or malonyl-CoA; and/or c) increase insulin sensitivity in adipose tissue, skeletal muscle, liver or pancreatic β cells; and/or d) modulate insulin secretion from pancreatic β cells.

ix) The above use wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

x) The use of a compound according to this invention for the preparation of a pharmaceutical composition for the treatment and/or prevention of dyslipidemia.

xi) The use of a compound according to this invention for the preparation of a pharmaceutical composition for the treatment and/or prevention of hyperlipidemia.

xii) The use of a compound according to this invention for the preparation of a pharmaceutical composition for the treatment and/or prevention of hyperglycemia.

xiii) The use of a compound according to this invention for lowering HbA_{1c}.

xiv) The use of a compound according to this invention for the preparation of a pharmaceutical composition for the treatment and/or prevention impaired glucose tolerance.

xv) The use of a compound according to this invention for the preparation of a pharmaceutical composition for the treatment and/or prevention of metabolic syndrome X.

xvi) The use of a compound according to this invention for the preparation of a pharmaceutical composition for the treatment and/or prevention of atheroschlerosis.

xvii) The use of a compound according to this invention for the preparation of a pharmaceutical composition for delaying or prevention of the progression from impaired glucose tolerance to diabetes type 2.

xviii) The use of a compound according to this invention for the preparation of a pharmaceutical composition for delaying or prevention of the progression from non-insulin requiring diabetes type 2 to insulin requiring diabetes type 2.

xix) The use according to any one of the above indications wherein a further antidiabetic, antiobesity, antihypertensive or appetite regulating drug is used.

xx) The use according to any one of the above indications, wherein metformin is also used.

xxi) The preparation of a pharmaceutical composition for the treatment and/or prevention of diabetes type 2.

xxii) A method of treating a disorder of a patient as described herein where modulation of the activity of hormone-sensitive lipase is desired, the method comprising administering to a subject in need thereof a therapeutically effective amount of a compound according to this invention or a pharmaceutically acceptable salt thereof.

xxiii) A method of treating a disorder of a patient as described herein where lowering of the activity of hormone-sensitive lipase is desired, the method comprising administering to a subject in need thereof a therapeutically effective amount of a compound according to this invention or a pharmaceutically acceptable salt thereof.

xxiv) The above methods wherein said administration is carried out by the oral, nasal, transdermal, pulmonal, or parenteral route.

xxv) The above methods wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

xxvi) Any one of the above methods wherein the therapeutically effective amount of the compound is from about 0.05 to about 2000 mg, preferably from about 0.1 to about 500 mg and even more preferable from about 1.0 to about 100 mg of said compound per day.

xxvii) Any one of the above methods wherein a further antidiabetic, antiobesity, antihypertensive or appetite regulating drug is administered to the patient.

xxviii) Any one of the above methods wherein metformin is also administered to the patient.

This invention also encompasses pharmaceutically acceptable salts of the compounds of formula I. Such salts include pharmaceutically acceptable acid addition salts, pharmaceutically acceptable salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoro-acetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxylnaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium, zinc, calcium salts and the like. Examples of amines and organic amines include ammonium, methylamine, dimethylamine, trimethylamine, ethylamine, diethylamine, propylamine, butylamine, tetramethylamine, ethanolamine, diethanolamine, triethanolamine, meglumine, ethylene-diamine, choline, N,N′-dibenzylethylenediamine, N-benzylphenylethylamine, N-methyl-D-glucamine, guanidine and the like. Examples of cationic amino acids include lysine, arginine, histidine and the like.

Acid addition salts wherever applicable are prepared by treatment with strong acids in solvents like ethyl acetate, ether, alcohols, acetone, THF, dioxane etc. Mixture of solvents may also be used.

Various polymorphs of compound of formula I forming part of this invention may be prepared by crystallization of compound of formula I under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

This invention also encompasses prodrugs of the compounds of formula I, which on administration undergo chemical conversion by metabolic processes before becoming active pharmacological substances. In general, such prodrugs will be functional derivatives of the compounds of formula I, which are readily convertible in vivo into the required compound of the formula I. Conventional procedures for the selection and preparation of suitable prodrug derivatives are described, for example, in “Design of Prodrugs”, ed. H. Bundgaard, Elsevier, 1985.

This invention also encompasses active metabolites of the compounds of formula I.

This invention also relates to pharmaceutical compositions comprising, as an active ingredient, at least one compound of the formula I or a pharmaceutically acceptable salt thereof together with one or more pharmaceutically acceptable carriers or diluents.

Furthermore, this invention relates to the use of compounds of formula I or their tautomeric forms, their stereoisomers, their polymorphs, their pharmaceutically acceptable salts or pharmaceutically acceptable solvates thereof for the preparation of a pharmaceutical composition for the treatment and/or prevention of disorders where a decreased level of plasma FFA is desirable, such as the conditions mentioned above.

In another aspect, this invention relates to a method of treating and/or preventing type 2 diabetes, insulin resistance, metabolic syndrome X, impaired glucose tolerance, dyslipidemia and abnormalities of lipoprotein metabolism.

In a still further aspect, this invention relates to the use of one or more compounds of formula I, or pharmaceutically acceptable salts thereof, for the preparation of a pharmaceutical composition for the treatment and/or prevention of type 2 diabetes, insulin resistance, metabolic syndrome X, impaired glucose tolerance, dyslipidemia and abnormalities of lipoprotein metabolism.

In a still further aspect, the compounds of formula I are useful for the delaying or prevention of the progression from impaired glucose tolerance to type 2 diabetes.

In a still further aspect, the compounds of formula I are useful for the delaying or prevention of the progression from non-insulin requiring type 2 diabetes to insulin requiring type 2 diabetes.

In another aspect, the compounds of formula I reduce triglyceride levels and are accordingly useful for the treatment and/or prevention of ailments and disorders such as diabetes and/or obesity.

In still another aspect, the compounds of formula I are useful for the treatment of hyperglycemia, elevated HbA_{1c} level, hyperinsulinemia, type 1.5 diabetes, latent autoimmune diabetes in adults, maturity onset diabetes, beta-cell apoptosis, hemochromatosis induced diabetes, impaired glucose tolerance, impaired fasting glucose, metabolic syndrome X, insulin resistance, impaired lipid tolerance, cystic fibrosis related diabetes, polycystic ovarian syndrome, and gestational diabetes.

In still another aspect, the compounds of formula I are useful for the treatment of obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, restenosis, intermittent claudication (atherosclerosis oblitterens), cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopy, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, and irritable bowel syndrome.

In still another aspect, the compounds of formula I may be useful for the treatment of diabetic retinopathy, background retinopathy, preproliferative retinopathy, proliferative retinopathy, macular edema, cataracts, nephropathy, nephrotic syndrome, diabetic nephropathy, microalbuminuria, macroalbuminuria, neuropathy, diabetic neuropathy, distal symmetrical sensorimotor polyneuropathy, and diabetic autonomic neuropathy.

In still another aspect, the compounds of formula I are useful for increasing the number of beta-cells in a patient, increasing the size of beta-cells in a patient or stimulating beta-cell proliferation, modulating beta-cell function and insulin secretion in a patient in need thereof, which method comprises administration of an effective amount of a compound of formula I to a patient in need thereof.

The compounds of this invention are also useful for reducing body weight in a patient in need thereof.

The compounds of this invention are also useful for weight neutral treatment of above mentioned diseases.

The compounds of this invention are also useful for redistributing fat in a patient in need thereof.

The compounds of this invention are also useful for redistributing central fat in a patient in need thereof.

The compounds of this invention are also useful for reducing or preventing central obesity.

The compounds of this invention are also useful for reducing postprandial serum lipid excursions.

The compounds of this invention are also useful for the treatment of fatty acid oxidation disorders such as MCAD.

In still another aspect, the compounds of formula I are useful for the treatment of a disease, condition or disorder wherein cholesterol is a precursor. Such diseases, conditions or disorders may relate to testosterone, for example, male contraception, excessive testosterone levels, PCOS and prostate cancer. They may also relate to cortisol or corticotropin, for example, Cushing disease.

The compounds of this invention are also useful for the treatment of cancer. Thus, the compounds of formula I may be useful for the treatment of insulinoma (pancreatic islet cell tumors), for example, malignant insulinomas and multiple insulinomas, adipose cell carcinomas, for example, lipocarconoma.

The compounds of this invention are also useful for the treatment of phaechromocytoma and other diseases with increased catecholamine incretion.

The compounds of this invention are also useful for the treatment of prostate cancer, for example, adenocarcinoma.

In still another aspect, the compounds of formula I may be used for the treatment of hepatic steatosis.

In still another aspect, the compounds of formula I may be used for the treatment of cirrhosis.

In still another aspect, the compounds of formula I may be used for the treatment of AIDS or an AIDS related diseases, condition or disorders In still another aspect, the compounds of formula I may be used for the treatment of lipodystrophy

In still another aspect, the compounds of formula I may be used for the treatment of lactic acidosis.

In yet another aspect, the compounds of this invention can be used to the treatment of CNS diseases, conditions or disorders.

Thus, the compound of this invention may be used for the treatment of Parkinson's disease, Alzheimers disease, ADHD (Attention Deficit Hyperactivity Disorder), feeding disorders such as bulimia and anorexia, depression, anxiety, cognitive memory disorders, age related cognitive decline, mild cognitive impairment and schizophrenia.

In yet another aspect, the compounds of this invention may be used for the treatment of inflammatory disorders, for example, rheumatoid arthritis, psoriasis, systemic inflammatory response syndrome, sepsis and the like.

The compounds of formula I may also be administered in combination with one or more further pharmacologically active substances, for example, selected from antiobesity agents, antidiabetics, antihypertensive agents, agents for the treatment and/or prevention of complications resulting from or associated with diabetes and agents for the treatment and/or prevention of complications and disorders resulting from or associated with obesity.

Thus, in a further aspect of this invention the compounds of formula I may be administered in combination with one or more antiobesity agents or appetite regulating agents. Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and nor- adrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators or TR β agonists.

In one embodiment of this invention, the antiobesity agent is leptin.

In another embodiment, the antiobesity agent is dexamphetamine or amphetamine.

In another embodiment, the antiobesity agent is fenfluramine or dexfenfluramine.

In still another embodiment, the antiobesity agent is sibutramine.

In a further embodiment, the antiobesity agent is orlistat.

In another embodiment, the antiobesity agent is mazindol or phentermine.

Suitable antidiabetics comprise insulin, exendin-4, GLP-1 (glucagon like peptide-1) and derivatives thereof such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as HMG CoA inhibitors (statins), compounds lowering food intake, RXR agonists and agents acting on the ATP-dependent potassium channel of the β-cells.

In one embodiment of this invention, the compounds of formula I are administered in combination with insulin.

In a further embodiment, the compounds of formula I are administered in combination with a sulphonylurea, for example, tolbutamide, glibenclamide, glipizide or glicazide.

In another embodiment, the compounds of formula I are administered in combination with a biguanide, for example, metformin.

In yet another embodiment, the compounds of formula I are administered in combination with a meglitinide, for example, repaglinide or senaglinide.

In a further embodiment, the compounds of formula I are administered in combination with an α-glucosidase inhibitor, for example, miglitol or acarbose.

In another embodiment, the compounds of formula I are administered in combination with an agent acting on the ATP-dependent potassium channel of the β-cells, for example, tolbutamide, glibenclamide, glipizide, glicazide or repaglinide.

Furthermore, the compounds of formula I may be administered in combination with nateglinide.

In still another embodiment, the compounds of formula I are administered in combination with an antihyperlipidemic agent or antilipidemic agent, for example, cholestyramine, colestipol, clofibrate, gemfibrozil, lovastatin, pravastatin, simvastatin, probucol or dextrothyroxine.

In a further embodiment, the compounds of formula I are administered in combination with more than one of the above-mentioned compounds, for example, in combination with a sulphonylurea and metformin, a sulphonylurea and acarbose, repaglinide and metformin, insulin and a sulphonylurea, insulin and metformin, insulin, insulin and lovastatin, etc.

Furthermore, the compounds of formula I may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds of formula I with one or more of the above-mentioned compounds and optionally one or more further pharmacologically active substances are considered to be within the scope of this invention.

The compounds of this invention may be administered alone or in combination with pharmaceutically acceptable carriers or excipients, in either single or multiple doses. The pharmaceutical compositions according to this invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. The compositions may appear in conventional forms, for example, capsules, tablets, aerosols, solutions, suspensions or topical applications.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of this invention.

Other suitable administration forms include suppositories, sprays, ointments, cremes, gels, inhalants, dermal patches, implants etc.

The therapeutic dose of the compound will depend upon the frequency and mode of administration, the sex, age, weight and general condition of the subject treated, the nature and severity of the condition treated and any concomitant diseases to be treated and other factors evident to those skilled in the art. The formulations may conveniently be presented in unit dosage form by methods known to those skilled in the art. In one embodiment, the composition in unit dosage form, comprises from about 0.05 to about 2000 mg, preferably from about 0.1 to about 500 mg of the compound of formula I pharmaceutically acceptable salt thereof.

In a still further embodiment, the pharmaceutical composition is for oral, nasal, transdermal, pulmonal, or parenteral administration.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of this invention contains a free base, such salts are prepared in a conventional manner by treating a solution or suspension of a free base of the compound with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the compounds of formula I in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents.

The pharmaceutical compositions formed by combining the compounds of this invention and the pharmaceutically acceptable carriers are then readily administered in a variety of dosage forms suitable for the disclosed routes of administration. The formulations may conveniently be presented in unit dosage form by methods known in the art of pharmacy.

Formulations of this invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain a core with the following constituents: 5 mg of active compound (as free compound or salt thereof), 1.5 mg of colloidal silicon dioxide (Aerosil), 70 mg of cellulose, microcrystalline (Avicel), 7.5 mg of modified cellulose gum (Ac-Di-Sol) and magnesium stearate (q.s.) with a coating of approximately 9 mg of HPMC and approximately 0.9 mg of Mywacett 9-40 T (acylated monoglyceride used as plasticizer for film coating).

The compounds of this invention may be administered to a patient which is a mammal, especially a human in need thereof. Such mammals include also animals, both domestic animals, for example, household pets, and non-domestic animals such as wildlife.

In a further aspect of this invention, the compounds of formula I may be administered in combination with further pharmacologically active substances, for example, an antidiabetic or other pharmacologically active material, including other compounds for the treatment and/or prevention of insulin resistance and diseases, wherein insulin resistance is the pathophysiological mechanism.

Furthermore, the compounds of formula I may be administered in combination with antiobesity agents or appetite regulating agents.

Pharmacological Methods

Compounds of formula I may be evaluated in vitro for their efficacy and potency to inhibit HSL, and such evaluation may be performed as described below.

Assays

Hormone-sensitive lipase (HSL)

Materials. The Hormone-sensitive lipase was provided by Dr. Cecilia Holm, from Lund University Sweden or produced and purified by Novo Nordisk (NN) using the reagents and protocols used by Dr. Holm. The substrates used are: ³H-labeled triolein (TO) from Amersham, Buckinghamshire, U.K. cat o. TRA191; 5-20 Ci/mmol dissolved in toluene, triolein (Sigma, Cat. No. T-1740), fluorochrome-labeled triacylglyceride (cis-octadec-9-enoic acid 2-[12-(7-nitrobenzo[1,2,5]oxadiazol-4-ylamino)-dodecanoyloxy]-1-cis-octadec-9-enoyloxymethylethyl ester) prepared by Novo Nordisk (NN) by conventional methods, and 1,3-(di[³H]-stearin), 2-(PEG-Biotin)glycerol prepared in collaboration with Amersham Pharmacia Biotech, UK and described in WO 01/073442. Phosphatidyl choline (PC) and phosphatidyl inositol (PI) are from Sigma (St Luis Mo. cat. Nos. P-3556 and P-5954, respectively). All other reagents are of commercial grade and obtained from various commercial sources.

Methods.

3190.1: Assay for determination of percent inhibition of hormone sensitive lipase by compound at 10μM sample concentration.

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 70 μL PED-BSA buffer) is pre-incubated for 30 min at 25° C. before addition of substrate (100 μL). Amount of formed product is measured after 120 min incubation at 37° C.

Results are given as percent activity relative to a non-inhibited sample (no compound).

3190.2: Assay for determination of IC₅₀ value for the inhibition of hormone sensitive lipase by compound. Standard concentrations of compound are 100 μM and 5-fold dilutions (initial concentration corresponding to 10 μM final concentration and 5-fold).

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 70 μL PED-BSA buffer) is pre-incubated for 30 min at 25° C. before addition of substrate (100 μL). Amount of formed product is measured after 120 min incubation at 37° C.

Results are given as IC₅₀ values after 4PL fit of obtained activity data.

The following table shows the IC₅₀ values of some compounds of formula I:

| Compound prepared in | |
|---|---|
| example number | IC₅₀ value, μM |
| 1 | 0.4 |
| 3 | 0.04 |
| 4 | 0.4 |

Abbreviations

In the examples below, the following terms are intended to have the following, general meanings: g is gram(s), h is hour(s), mg is milligram(s), MHz is megahertz, min is minute(s), mmol is millimole(s), mL is milliliter(s), ppm is parts per million, psi is pounds per square inch, APCI is atmospheric pressure chemical ionization, ESI is electrospray ionization, m/z is mass to charge ration, Mp is melting point, MS is mass spectroscopy, HPLC is high performance liquid chromatography, RP is reverse phase, HPLC-MS is high performance liquid chromatography mass spectroscopy, NMR is nuclear magnetic resonance spectroscopy, tᵣ is retention time, DMSO-d₆ is hexadeuterio dimethylsulfoxide.

HPLC-MS

The following instrumentation was used:

Hewlett Packard series 1100 G1312A Bin Pump

Hewlett Packard series 1100 Column compartment

Hewlett Packard series 1100 G13 15A DAD diode array detector

Hewlett Packard series 1100 MSD

Sedere 75 Evaporative Light Scattering detector

The instrument was controlled by HP Chemstation software.

The HPLC pump was connected to two eluent reservoirs containing:

| | |
|---|---|
| A: | 0.05% TFA in water |
| B: | 0.05% TFA in acetonitrile |

The analysis was performed at 40° C. by injecting an appropriate volume of the sample (preferably 1 μl) onto the column, which is eluted with a gradient of acetonitrile.

After the DAD the flow is divided yielding approximately 1 mL/min to the ELS and 0.5 mL/min to the MS.

The HPLC conditions, detector settings and mass spectrometer settings which were used are as follows:

Method A:

| | |
|---|---|
| Column | Waters Xterra MS C₁₈ 5 μm 3 mm id × 50 mm |
| Gradient | 5%-100% acetonitrile linear during 7.5 min at 1.5 ml/min |
| Detection | 210 nm (analogue output from DAD) |
| | ELS (analogue output from ELS) |
| MS | Ionization mode API-ES, Scan 100-1000 amu step 0.1 amu |

Method B:

| | |
|---|---|
| Column | Waters Xterra MS C₁₈ 5 μm 3 mm id × 50 mm |
| Gradient | 5%-95% acetonitrile linear during 3.5 min at 2.7 ml/min |
| Detection | 210 nm (analogue output from DAD) |
| | ELS (analogue output from ELS) |
| MS | Ionisation mode API-ES, Scan 100-1000 amu step 0.1 amu |

### EXAMPLE 1

3,4-Dihydro-1H-isoquinoline-2-carboxylic acid 4,4-dimethyl-2,6-dioxo-3,4,5,6-tetrahydro-2H-[1,3′]bi-pyridinyl-6′-yl ester

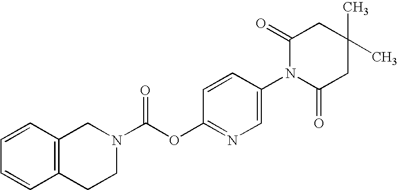

3,4-Dihydro-1H-isoquinoline-2-carbonyl chloride (7.83 g, 40.0 mmol) was added to a stirred solution of 6′-hydroxy-4,4-dimethyl-4,5-dihydro-3H-[1,3′]bipyridinyl-2,6-dione (9.37 g, 40.0 mmol) and 1,4-diazabi-cyclo[2.2.2]octane (4.49 g, 40.0 mmol) in N,N-dimethylformamide (50 mL). After stirring for 1.5 h, the solution was filtered and water was added to the filtrate. The yellow precipitate was isolated by suction and dried in a vacuum oven. Crystallization from ethyl acetate/heptane yielded the title compound (9.68 g, 62% yield). Mp: 156-158° C.
¹NMR (400 MHz, CDCl₃) δ 1.22 (s, 6H), 2.70 (s, 4H), 2.97 (q, 2H), 3.82 (t, 1H), 3.91 (t, 1H), 4.73 (s, 1H), 4.87 (s, 1H), 7.11-7.29 (m, 5H), 7.52 (dd, 1H), 8.11 (d, 1H); HPLC-MS (Method A): m/z=394 (M+H)⁺; tᵣ=3.91 min.

### EXAMPLE 2

6,7-Dimethoxy-3,4-dihydro-1H-isoquinoline-2-carboxylic acid 4,4-dimethyl-2,6-dioxo-3,4,5,6-tetra-hydro-2H-[1,3′]bipyridinyl-6′-yl ester

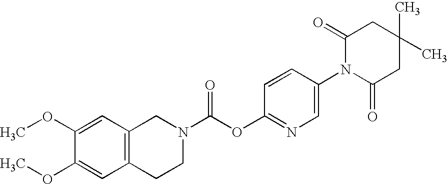

Phosgene (20% in toluene, 5 mL) is slowly added by means of syringe to a stirred solution of 6′-hydroxy-4,4-dimethyl -4,5-dihydro-3H-[1,3′]bipyridinyl-2,6-dione (234 mg, 1.00 mmol) and N,N,-diiso-propylethylamine (0.19 g, 1.1 mmol) in dichloromethane. After stirring for 1½ h at room temperature the solvent is evaporated in vacuo and the residue is redissolved in dichloromethane. At 0° C., this solution is slowly added to a solution of 6,7-dimethoxy-1,2,3,4-tetrahydroisoquinoline hydrochloride (213 mg, 0.92 mmol) and 1,4-diazabicyclo[2.2.2]octane (0.11 g, 1.00 mmol) in dichloromethane (4 25 mL). After stirring overnight the solution is extracted twice with water. The dichloromethane layer is evaporated and the residue purified by preparative HPLC. Recrystallisation from ethyl acetate yielded the title compound (10 mg, 2.4% yield).
¹H NMR (400 MHz, CDCl₃) δ1.22 (s, 6H), 2.70 (s, 4H), 2.88 (q, 2H), 3.80 (t, 1H), 3.86 (s, 3H), 3.88 (s, 3H), 3.90 (t, 1H), 4.67 (s, 1H), 4.79 (s, 1H), 6.62 (d, 1H),6.67 (s, 1H), 7.28 (m, 1H), 7.52 (dd, 1H), 8.11 (d, 1H); HPLC-MS (Method A): m/z=454 (M+H)⁺; tᵣ=3.24 min.

### EXAMPLE 3

3,4-Dihydro-1H-isoquinoline-2-carboxylic acid 5-(7,9-dioxo-8-azaspiro[4.5]dec-8-yl)pyridin-2-yl ester

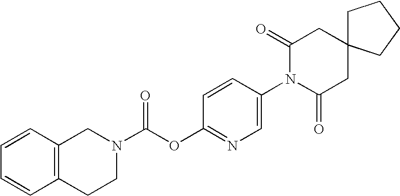

### Step A:

4,4-Tetramethyleneglutaric anhydride (25 g, 149 mmol) was added to a stirred solution of 5-amino-2-methoxypyridine (18.45 g, 149 mmol) in dichloromethane (150 mL). After stirring for 3 h at room temperature thionyl chloride (16.2 mL, 1.5 equiv.) was added slowly. After stirring for 3.5 h at room temperature, diethyl ether (500 mL) was added and the pink solids were isolated by suction, washed thoroughly with diethyl ether and dried overnight in a vacuum oven, yielding 8-(6-methoxypyridin-3-yl)-8-azaspiro[4.5]decane-7,9-dione hydrochloride (46.5 g, 101% yield).
¹H NMR (400 MHz, DMSO-d₆) δ1.55 (m, 4H), 1.68 (m, 4H), 2.77 (s, 4H), 3.89 (s, 3H), 6.91 (d, 1H), 7.50 (dd, 1H), 7.92 (d, 1H), 9.12 (br.s, 1H); HPLC-MS (Method B): m/z=275 (M+H)⁺; tᵣ=1.45 min.

### Step B:

8-(6-Methoxypyridin-3-yl)-8-azaspiro[4.5]decane-7,9-dione hydrochloride was heated in a kugelrohr oven at 180° C. for 10-15 minutes. The crude 8-(6-hydroxypyridin-3-yl)-8-azaspiro[4.5]decane-7,9-dione was used in the next step without further purification.
hu 1H NMR (400 MHz, DMSO-d₆) δ1.52 (m, 4H), 1.67 (m, 4H), 2.70 (s, 4H), 6.33 (d, 1H), 7.18 (dd, 1H), 7.30 (d, 1H), 11.73 (br.s, 1H); HPLC-MS (Method B): m/z=261 (M+H)⁺; tᵣ=1.01 min.

### Step C:

3,4-Dihydro-1H-isoquinoline-2-carbonyl chloride (8.61 g, 44.0 mmol) was added to a mixture of 8-(6-hydroxypyridin-3-yl) -8-azaspiro[4.5]decane-7,9-dione (10.41 g, 40.0 mmol) and 1,4-diazabicyclo-[2.2.2]octane (4.94 g, 44.0 mmol) in N,N-dimethylformamide (50 mL). After stirring overnight at room temperature water was added and the solid material was isolated by suction. The solid was dissolved in dichloromethane, dried over sodium sulfate, filtered and evaporated in vacuo. The residue was recrystallised from ethyl acetate/heptane followed by a second crystallization from pure ethyl acetate yielding the title compound (7.92 g, 47% yield).
¹H NMR (400 MHz, CDCl₃) δ1.65 (m, 4H), 1.79 (m, 4H), 2.79 (s, 4H), 2.97 (q, 2H), 3.82 (t, 1H), 3.91 (t, 1H), 4.73 (s, 1H), 4.86 (s, 1H), 7.10-7.30 (m, 5H), 7.51 (dd, 1H), 8.10 (d, 1H).; HPLC-MS (Method A): m/z=420 (M+H)⁺; tᵣ=3.71 min.

### EXAMPLE 4

3,4-Dihydro-1H-isoquinoline-2-carboxylic acid 5-(3-thia-1-azaspiro[4.4] non-1-en-2-ylamino)-pyridin-2-yl ester

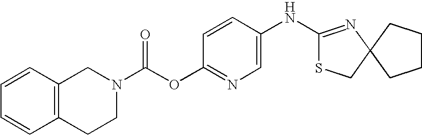

### Step A:

A solution of 5-nitro-2-(2-trimethylsilanylethoxy)pyridine (9.78 g, 40.7 mmol) in ethyl acetate (50 mL) was hydrogenated with a catalytic amount of 10% Pd/C in a Parr-apparatus at 40 psi H₂-pressure during 5 h. The catalyst was removed by filtration over Celite and the solvent was removed in vacuo leaving 6-(2-trimethylsilanylethoxy)pyridin-3-ylamine (8.14 g, 95% yield) as an oil.
¹H NMR (400 MHz, CDCl₃) δ1.03 (m, 2H), 3.32 (br.s, 2H), 4.22 (m, 2H), 6.50 (d, 1H), 6.95 (dd, 1H), 7.59 (d, 1H); HPLC-MS (Method A): m/z=183 (M-CH₂CH₂+H)⁺, 211 (M+H)⁺; tᵣ=2.54 min.

### Step B:

At 0° C., 6-(2-trimethylsilanylethoxy)pyridin-3-ylamine (3.00 g, 14.26 mmol) was added to a stirred solution of di-2-pyridyl thionocarbonate (3.32 g, 14.26 mmol) in dichloromethane (40 mL). After stirring at room temperature for 2 h (1-amino-1-cyclopentyl)methanol (1.64 g, 14.26 mmol) dissolved in a small amount of dichloromethane was added in one portion. After stirring overnight most of the solvent was removed by evaporation in vacuo and the residue was purification by flash column chromatography (SiO₂, ethyl acetate/heptane 3:7). The product was stirred with some heptane, filtrated and dried overnight in vacuum oven at 40° C., yielding 1-(1-hydroxymethyl-yclopentyl)-3-[6-(2-trimethylsilanylethoxy) pyridin-3-yl]thiourea (3.44 g, 66% yield).
¹H NMR (400 MHz, CDCl₃) δ0.07 (s, 9H), 1.12 (m, 2H),1.61-1.99 (m, 9H), 3.79 (s, 2H), 4.35 (t, 2H),6.22 (br.s, 1H), 6.73 (d, 1H), 7.87 (br.s, 1H), 7.97 (d, 1H); HPLC-MS (Method B): m/z=368 (M+H)⁺; tᵣ=2.03 min.

### Step C:

At −20° C., thionyl chloride (1.19 ml, 16.32 mmol) was added to a solution of 1-(1-hydroxymethylcyclo-pentyl)-3-[6-(2-trimethylsilanylethoxy)pyridin-3-yl]thiourea (3.00 g, 8.16 mmol) in dichloromethane (10 ml). Stirring was continued at −20° C. for 30 min. Some extra dichloromethane was added. The solids were isolated by suction and dried overnight in a vacuum oven at 40° C., yielding (3-thia-1-azaspiro-[4.4]non-1-en-2-ylamino)-[6-(2-trimethylsilanylethoxy)pyridine-3-yl]amine, which was used in the next step without further purification.

### Step D:

Trifluoroacetic acid (0.5 mL) was added to a suspension of (3-thia-1-azaspiro[4.4]non-1-en-2-ylamino)-[6-(2-trimethylsilanylethoxy)pyridine-3-yl]amine in dichloromethane (50 mL). After stirring for 3 h the solvent is evaporated in vacuo and the residue is dried in vacuum oven at 50° C., yielding 5-(3-thia-1-azaspiro[4.4]non-1-en-2-ylamino)pyridin-2-ol (1.1 g, 54% yield).
¹H NMR (400 MHz, CDCl₃) δ1.77 (m, 2H), 1.97 (m, 4H), 2.12 (m, 2H), 3.41 (s, 1H), 6.25 (d, 1H), 7.59 (m, 2H), 12.45 (br.s, 1H), 12.73 (br.s, 1H); HPLC-MS (Method B): m/z=250 (M+H)⁺; tᵣ=0.84 min.

### Step E:

3,4-Dihydro-1H-isoquinoline-2-carbonyl chloride (117 mg, 0.60 mmol) was added to a solution of 5-(3-thia-1-aza-spiro[4.4]non-1-en-2-ylamino)pyridin-2-ol (0.10 g, 0.4 mmol) and 1,4-diazabicyclo[2.2.2]-octane (0.7 g, 0.6 mmol) in N,N-dimethylformamide (2 mL). The solution was stirred for 3 h at room temperature. Purification by flash column chromatography (SiO₂, dichloromethane followed by ethyl acetate/dichloromethane 1:4) yielded the title compound (40 mg, 30% yield).
¹H NMR (400 MHz, CDCl₃) δ1.67-1.92 (m, 8H), 2.95 (m, 2H), 3.24 (s, 2H), 3.81 (t, 1H), 3.91 (t, 1H), 4.72 (s, 1H), 4.87 (s, 1H), 7.07 (d, 1H), 7.10-7.22 (m, 4H), 7.49 (d, 1H), 8.10 (d, 1H); HPLC-MS (Method A): m/z=409 (M+H)⁺; tᵣ=2.83 min.
The starting material in step A in this example has been synthesized according to Christos Papageorgiou, Gian Camenisch and Xaver Borer, Bioorg. Med. Chem. Lett. 2001, 11 (12), 1549-1552.
The “spiro thiazoline” in this example has been synthesized according to a slightly modified procedure as described by P. W. Manley and U. Quast, J. Med. Chem. 1992, 35, 2327-2340.
Analogously as described above, the following 42 compounds of formula I can be prepared:
[A] 7-Bromo-3,4-dihydro-1H-isoquinoline-2-carboxylic acid 4,4-dimethyl-2,6-dioxo-3,4,5,6-tetra-hydro -2H-[1,3′]bipyridinyl-6′-yl ester having the formula:

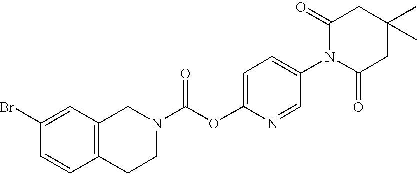

[B] 3,4-Dihydro-1H-isoquinoline-2-carboxylic acid 5-(2,4-dioxo-3-aza-spiro[5.5]undec-3-yl)-pyridin -2-yl ester having the formula:

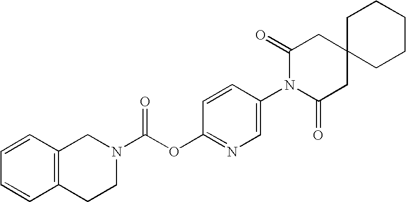

[C] 3,4-Dihydro-1H-isoquinoline-2-carboxylic acid 4,4-diethyl-2,6-dioxo-3,4,5,6-tetrahydro-2H-[1,3′]bipyridinyl-6′-yl ester having the formula:

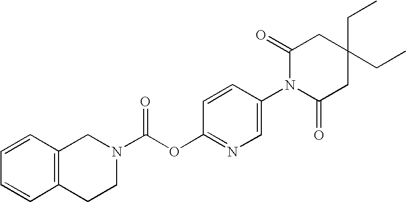

[D] 3,4-Dihydro-1H-isoquinoline-2-carboxylic acid 4-ethyl-4-methyl-2,6-dioxo-3,4,5,6-tetrahydro-2H-[1,3′]bipyridinyl-6′-yl ester having the formula:

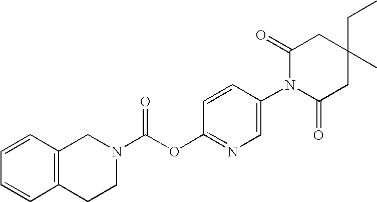

[E] 7-Chloro-3,4-dihydro-1H-isoquinoline-2-carboxylic acid 5-(7,9-dioxo-8-aza-spiro[4.5]dec-8-yl)pyridin-2-yl ester having the formula:

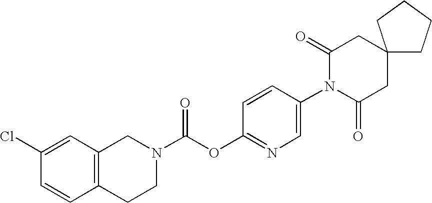

[F] 7-Fluoro-3,4-dihydro-1H-isoquinoline-2-carboxylic acid 5-(7,9-dioxo-8-aza-spiro[4.5]dec-8-yl)pyridin-2-yl ester having the formula:

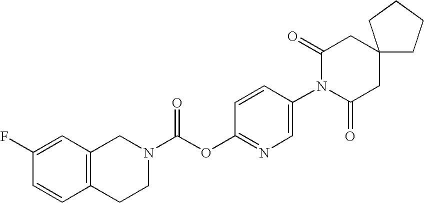

[G] 6-Chloro-3,4-dihydro-1H-isoquinoline-2-carboxylic acid 5-(7,9-dioxo-8-aza-spiro[4.5]dec-8-yl)pyridin-2-yl ester having the formula:

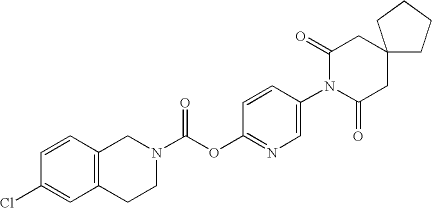

[H] 6-Fluoro-3,4-dihydro-1H-isoquinoline-2-carboxylic acid 5-(7,9-dioxo-8-aza-spiro[4.5]dec-8-yl)pyridin-2-yl ester having the formula:

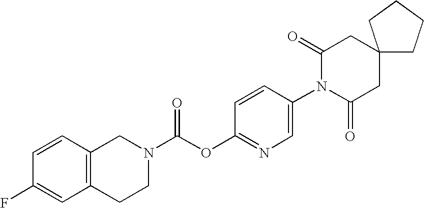

[I] 7-Chloro-3,4-dihydro-1H-isoquinoline-2-carboxylic acid 4,4-dimethyl-2,6-dioxo-3,4,5,6-tetra-hydro-2H-[1,3′]bipyridinyl-6′-yl ester having the formula:

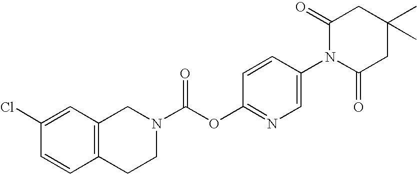

[J] 7-Fluoro-3,4-dihydro-1H-isoquinoline-2-carboxylic acid 4,4-dimethyl-2,6-dioxo-3,4,5,6-tetra-hydro-2H-[1,3′]bipyridinyl-6′-yl ester having the formula:

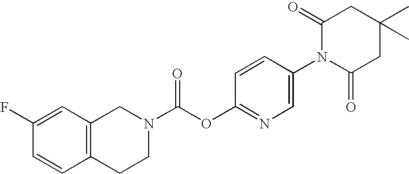

[K] 6-Chloro-3,4-dihydro-1H-isoquinoline-2-carboxylic acid 4,4-dimethyl-2,6-dioxo-3,4,5,6-tetra-hydro-2H-[1,3′]bipyridinyl-6′-yl ester having the formula:

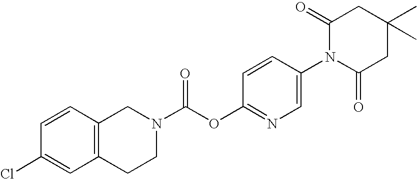

[L] 6-Fluoro-3,4-dihydro-1H-isoquinoline-2-carboxylic acid 4,4-dimethyl-2,6-dioxo-3,4,5,6-tetra-hydro-2H-[1,3′]bipyridinyl-6′-yl ester having the formula:

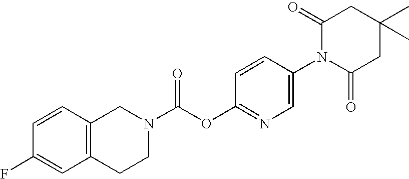

[M] 3,4-Dihydro-1H-isoquinoline-2-carboxylic acid 5-(4,4-dimethyl-4,5-dihydrothiazol-2-ylam ino)-pyridin-2-yl ester having the formula:

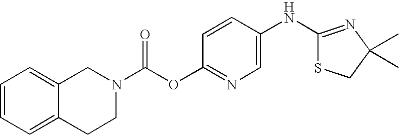

[N] 3,4-Dihydro-1H-isoquinoline-2-carboxylic acid 5-(4,4-diethyl-4,5-dihydrothiazol-2-ylamino)-pyridin-2-yl ester having the formula:

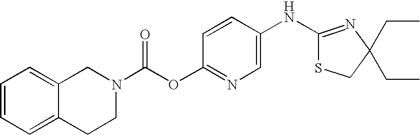

[O] 3,4- Dihydro-1H-isoquinoline-2-carboxylic acid 5-(4-ethyl-4-methyl-4,5-dihydrothiazol-2-yl-amino)pyridin-2-yl ester having the formula:

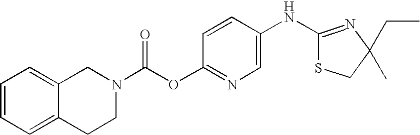

[P] 3,4-Dihydro-1H-isoquinoline-2-carboxylic acid 5-(4-methyl-2,6-dioxopiperazin-1-yl)-pyridin-2-yl ester having the formula:

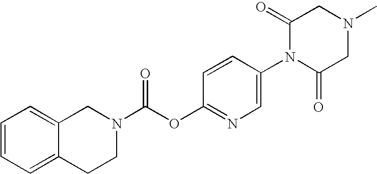

[Q] 3,4-Dihydro-1H-isoquinoline-2-carboxylic acid 5-(4-ethyl-2,6-dioxopiperazin-1-yl)-pyridin-2-yl ester having the formula:

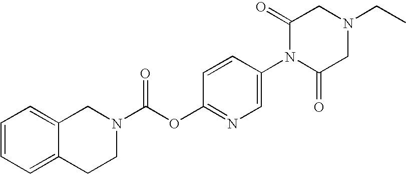

[R] 3,4-Dihydro-1H-isoquinoline-2-carboxylic acid 5-(4-isobutyl-2,6-dioxopiperazin-1-yl)-pyridin-2-yl ester having the formula:

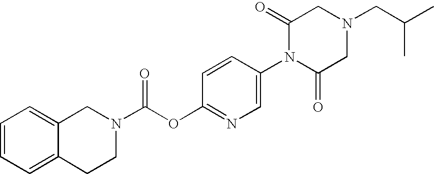

[S] 3,4-Dihydro-1H-isoquinoline-2-carboxylic acid 4,4-dimethyl-2-oxo-3,4,5,6-tetrahydro-2H-[1,3′]bipyridinyl-6′-yl ester having the formula:

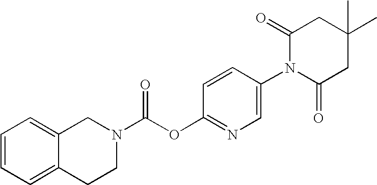

[T] 7-Chloro-3,4-dihydro-1H-isoquinoline-2-carboxylic acid 4,4-dimethyl-2-oxo-3,4,5,6-tetra-hydro-2H-[1,3′]bipyridinyl-6′-yl ester having the formula:

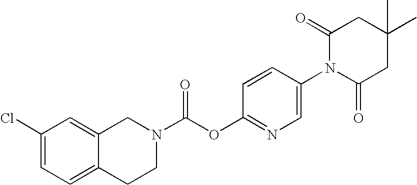

[U] 7-Fluoro-3,4-dihydro-1H-isoquinoline-2-carboxylic acid 4,4-dimethyl-2-oxo-3,4,5,6-tetra-hydro-2H-[1,3′]bipyridinyl-6′-yl ester having the formula:

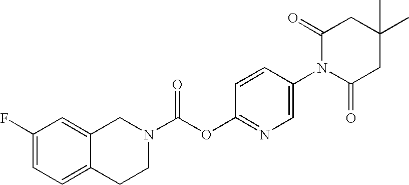

[V] 6-Chloro-3,4-dihydro-1H-isoquinoline-2-carboxylic acid 4,4-dimethyl-2-oxo-3,4,5,6-tetra-hydro-2H-[1,3′]bipyridinyl-6′-yl ester having the formula:

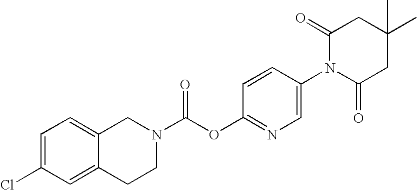

[W] 6- Fluoro-3,4-dihydro-1H-isoquinoline-2-carboxylic acid 4,4-dimethyl-2-oxo-3,4,5,6-tetra-hydro-2H-[1,3′]bipyridinyl-6′-yl ester having the formula:

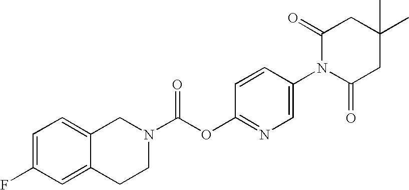

[X] 6,7-Dimethoxy-3,4-dihydro-1 H-isoquinoline-2-carboxylic acid 4,4-dimethyl-2-oxo-3,4,5,6-tetrahydro-2H-[1,3′]bipyridinyl-6′-yl ester having the formula:

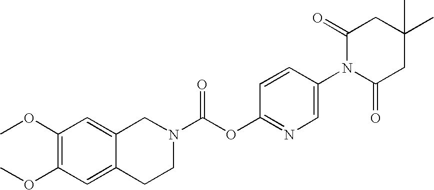

[Y] 7-Methoxy-3,4-dihydro-1H-isoquinoline-2-carboxylic acid 4,4-dimethyl-2-oxo-3,4,5,6-tetra-hydro-2H-[1,3′]bipyridinyl-6′-yl ester having the formula:

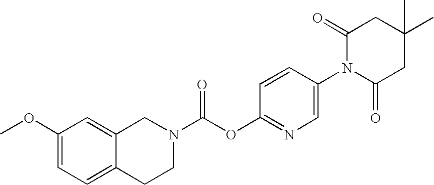

[Z] 6-Methoxy-3,4-dihydro-1H-isoquinoline-2-carboxylic acid 4,4-dimethyl-2-oxo-3,4,5,6-tetra-hydro-2H-[1,3′]bipyridinyl-6′-yl ester having the formula:

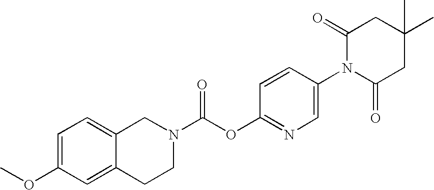

[AA] 3,4-Dihydro-1H-isoquinoline-2-carboxylic acid 4-isopropyl-2,6-dioxo-3,4,5,6-tetrahydro-2H-[1,3′]bipyridinyl-6′-yl ester having the formula:

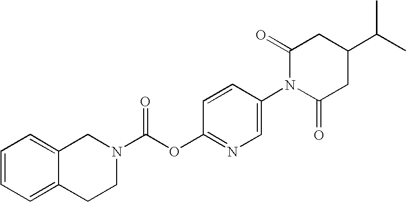

[BB] 7-Chloro-3,4-dihydro-1H-isoquinoline-2-carboxylic acid 4-isopropyl-2,6-dioxo-3,4,5,6-tetra-hydro-2H-[1,3′]bipyridinyl-6′-yl ester having the formula:

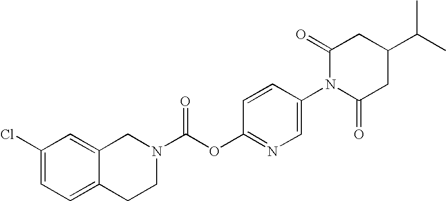

[CC] 7- Fluoro-3,4-dihydro-1H-isoquinoline-2-carboxylic acid 4-isopropyl-2,6-dioxo-3,4,5,6-tetra-hydro-2H-[1,3′]bipyridinyl-6′-yl ester having the formula:

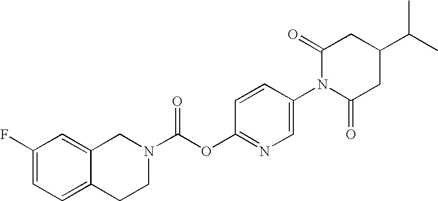

[DD] 6-Chloro-3,4-dihydro-1H-isoquinoline-2-carboxylic acid 4-isopropyl-2,6-dioxo-3,4,5,6-tetra-hydro-2H-[1,3′]bipyridinyl-6′-yl ester having the formula:

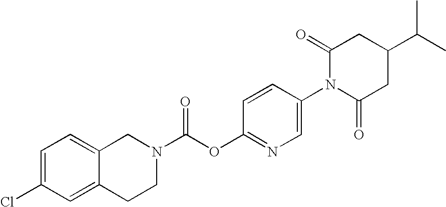

[EE] 6-Fluoro-3,4-dihydro-1H-isoquinoline-2-carboxylic acid 4-isopropyl-2,6-dioxo-3,4,5,6-tetra-hydro-2H-[1,3′]bipyridinyl-6′-yl ester having the formula:

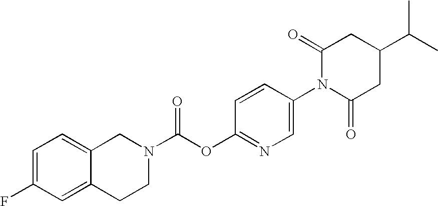

[FF] 6,7-Dimethoxy-3,4-dihydro-1H-isoquinoline-2-carboxylic acid 4-isopropyl-2,6-dioxo-3,4,5,6-tetrahydro-2H-[1,3′]bipyridinyl-6′-yl ester having the formula:

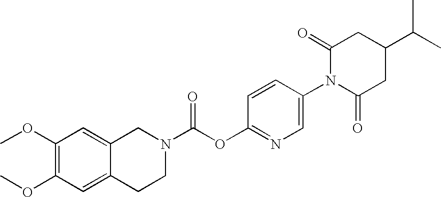

[GG] 7-Methoxy-3,4-dihydro-1H-isoquinoline-2-carboxylic acid 4-isopropyl-2,6-dioxo-3,4,5,6-tetra-hydro-2H-[1,3′]bipyridinyl-6′-yl ester having the formula:

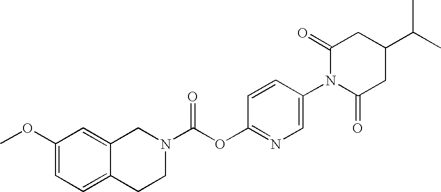

[HH] 6-Methoxy-3,4-dihydro-1H-isoquinoline-2-carboxylic acid 4-isopropyl-2,6-dioxo-3,4,5,6-tetra-hydro-2H-[1,3′]bipyridinyl-6′-yl ester having the formula:

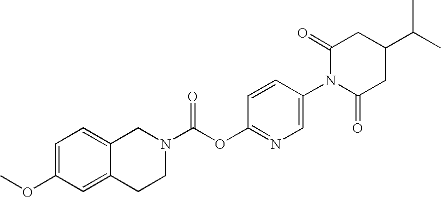

[II] 3,4-Dihydro-1H-isoquinoline-2-carboxylic acid 3,3-dimethyl-2,6-dioxo-3,4,5,6-tetrahydro-2H-[1,3′]bipyridinyl-6′-yl ester having the formula:

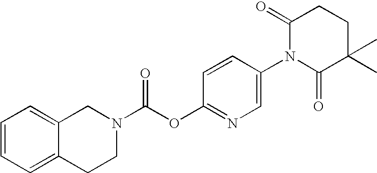

[JJ] 7-Chloro-3,4-dihydro-1H-isoquinoline-2-carboxylic acid 3,3-dimethyl-2,6-dioxo-3,4,5,6-tetra-hydro-2H-[1,3′]bipyridinyl-6′-yl ester having the formula:

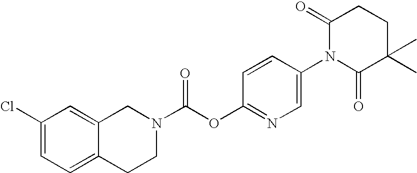

[KK] 7-Fluoro-3,4-dihydro-1H-isoquinoline-2-carboxylic acid 3,3-dimethyl-2,6-dioxo-3,4,5,6-tetra-hydro-2H-[1,3′]bipyridinyl-6′-yl ester having the formula:

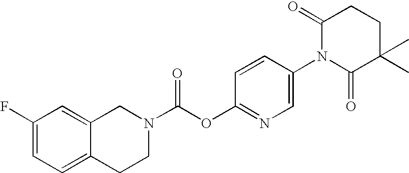

[LL] 6-Chloro-3,4-dihydro-1H-isoquinoline-2-carboxylic acid 3,3-dimethyl-2,6-dioxo-3,4,5,6-tetra-hydro-2H-[1,3′]bipyridinyl-6′-yl ester having the formula:

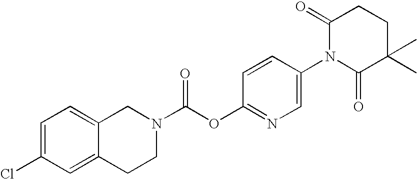

[MM] 6-Fluoro-3,4-dihydro-1H-isoquinoline-2-carboxylic acid 3,3-dimethyl-2,6-dioxo-3,4,5,6-tetra-hydro-2H-[1,3′]bipyridinyl-6′-yl ester having the formula:

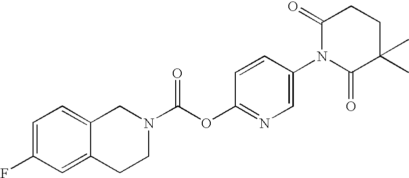

[NN] 6,7-Dimethoxy-3,4-dihydro-1H-isoquinoline-2-carboxylic acid 3,3-dimethyl-2,6-dioxo-3,4,5,6-tetrahydro-2H-[1,3′]bipyridinyl-6′-yl ester having the formula:

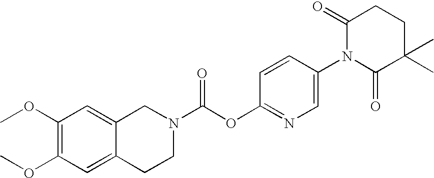

[OO] 7-Methoxy-3,4-dihydro-1H-isoquinoline-2-carboxylic acid 3,3-dimethyl-2,6-dioxo-3,4,5,6-tetrahydro-2H-[1,3′]bipyridinyl-6′-yl ester having the formula:

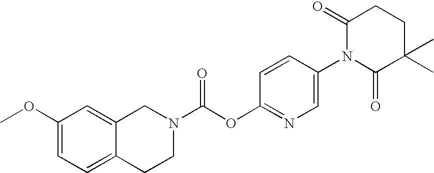

[PP] 6-Methoxy-3,4-dihydro-1H-isoquinoline-2-carboxylic acid 3,3-dimethyl-2,6-dioxo-3,4,5,6-tetrahydro-2H-[1,3′]bipyridinyl-6′-yl ester having the formula:

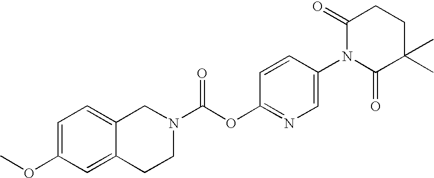

1. A compound of formula I 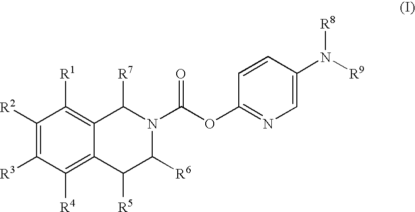
wherein R¹, R², R³, R⁴, R⁵, R⁶, and R⁷, independent of each other, each represents hydrogen, hydroxy, mercapto, amino, —CONH₂, —CSNH₂, —NH—CO—NH₂, —NH—CS—NH₂, halogen, —S(═O)₂(OH), C₁₋₆-alkyl, C₁₋₆-alkoxy, C₂₋₆-alkenyl, aryl, heteroaryl, C₃₋₈-heterocyclyl and C₃₋₁₃-cycloalkyl, wherein each of hydroxy, mercapto, amino, —CONH₂, —NH—CO—NH₂, —NH—CS—NH₂, —CSNH₂, —S(═O)₂(OH), C₁₋₆-alkyl, C₁₋₆-alkoxy, C₂₋₆-alkenyl, aryl, heteroaryl, C₃₋₈-heterocyclyl and C₃₋₁₃-cycloalkyl may optionally be substituted with one or more substituents independently selected from hydroxy, mercapto, oxo (═O), thioxo (═S), halogen, amino, —S(═O)₂(OH), C₁₋₆-alkyl, C₁₋₆-alkoxy, C₂₋₆-alkenyl, aryl, heteroaryl, C₃₋₈-heterocyclyl, and C₃₋₁₃-cycloalkyl, wherein each of hydroxy, mercapto, —S(═O)₂(OH), C₁₋₆-alkyl, C₁₋₆-alkoxy, C₂₋₆-alkenyl, aryl, heteroaryl, C₃₋₈-heterocyclyl and C₃₋₁₃-cycloalkyl may optionally be substituted with one or more substituents independently selected from hydroxy, mercapto, oxo, halogen, amino, —S(═O)₂(OH), halo-C₁₋₄-alkyl, halo-C₁₋₄-alkoxy, C₁₋₆-alkyl, C₁₋₆-alkoxy, C₂₋₆-alkenyl, aryl, heteroaryl, C₃₋₈-heterocyclyl, and C₃₋₁₃-cycloalkyl; and either R⁸ is hydrogen and R⁹ represents C₃₋₈-heterocyclyl which, optionally, is substituted with one or more substituents independently selected from hydroxy, mercapto, oxo (═O), thioxo (═S), halogen, amino, —S(═O)₂(OH), C₁₋₆-alkyl, C₁₋₆-alkoxy, C₂₋₆-alkenyl, aryl, heteroaryl, C₃₋₈-heterocyclyl, and C₃₋₁₃-cycloalkyl; or R⁸ together with R⁹ and together with the adjacent nitrogen atom represents C₃₋₈-heterocyclyl which, optionally, is substituted with one or more substituents independently selected from hydroxy, mercapto, oxo (═O), thioxo (═S), halogen, amino, —S(═O)₂(OH), C₁₋₆-alkyl, C₁₋₆-alkoxy, C₂₋₆-alkenyl, aryl, heteroaryl, C₃₋₈-heterocyclyl, and C₃₋₁₃-cycloalkyl; or a pharmaceutically acceptable salt thereof, or a pharmaceutically acceptable solvate thereof, tautomeric forms, stereoisomers, mixture of stereoisomers, racemic mixture, or polymorphs thereof.

2. canceled

3. canceled

4. canceled

5. canceled

6. canceled

7. canceled

8. canceled

9. canceled

10. canceled

11. canceled

12. canceled

13. canceled

14. canceled

15. The compound according to claim 1, wherein R¹ is hydrogen.

16. The compound according to claim 1, wherein R² is selected from hydrogen, alkoxy and halogen.

17. The compound according to claim 16, wherein R² is selected from hydrogen and alkoxy.

18. The compound according to claim 1, wherein R³ is selected from hydrogen, halogen and alkoxy.

19. The compound according to claim 18, wherein R³ is selected from hydrogen and alkoxy.

20. The compound according to claim 1, wherein R⁴ is hydrogen.

21. The compound according to claim 1, wherein R⁵ is hydrogen.

22. The compound according to claim 1, wherein R⁶ is hydrogen.

23. The compound according to claim 1, wherein R⁷ is hydrogen.

24. The compound according to claim 1, wherein R⁸ is hydrogen, and R⁹ is 4,5-dihydrothiazolyl substituted with one or two alkoxy groups in the thiazole ring.

25. The compound according to claim 1, wherein R⁹ is C₃₋₈-heterocyclyl, optionally substituted by C₃₋₁₃-cycloalkyl.

26. The compound according to claim 25, wherein R⁹ is 3-thia-1-azaspiro[4.4]non-1-en-2-yl.

27. The compound according to claim 1, wherein R⁸ and R⁹ together with the adjacent nitrogen atom is C₃₋₈-heterocyclyl, optionally substituted with one or more oxo, C₁₋₆-alkyl or C₃₋₁₃-cycloalkyl.

28. The compound according to claim 27, wherein R⁸ and R⁹ together with the adjacent nitrogen atom is selected from piperidino (1-piperidyl) or piperazinyl, each of which is optionally substituted with one of more of the following groups oxo and alkyl, wherein two alkyl substituents in the same position in the piperidino or piperazinyl ring may together form a ring.

29. The compound according to claim 28, wherein R⁸ and R⁹ together with the adjacent nitrogen atom is selected from 4,4-dimethyl-2,6-dioxo-3,4,5,6-tetrahydro-2H-pyridinyl; 2,4-dioxo-3-aza-spiro[5.5]undec-3-yl; 4,4-diethyl-2,6-dioxo-3 ,4,5,6-tetrahydro-2H-pyridinyl; 4-ethyl-4-methyl-2,6-dioxo-3,4,5,6-tetrahydro-2H-pyridinyl; 7,9-dioxo-8-aza-spiro[4.5]dec-8-yl; 4,4-dimethyl-2,6-dioxo-3 ,4,5,6-tetrahydro-2H-pyridinyl; 4-methyl-2,6-dioxopiperazin-1-yl; 4-ethyl-2,6-dioxopiperazin-1-yl; 4-isobutyl-2,6-dioxopiperazin-1-yl; 4,4-dimethyl-2-oxo-3,4,5,6-tetrahydro-2H-pyridinyl; 4-isopropyl-2,6-dioxo-3,4,5,6-tetrahydro-2H-pyridinyl; and 3,3-dimethyl-2,6-dioxo-3,4,5,6-tetrahydro-2H-pyridinyl.

30. The compound according to claim 29, wherein R⁸ and R⁹ together with the adjacent nitrogen atom is selected from 4,4-dimethyl-2,6-dioxo-3,4,5,6-tetrahydro-2H-pyridinyl and 7,9-dioxo-8-azaspiro[4.5]dec-8-yl.

31. The compound according to claim 1, selected from the following: 3,4-dihydro-1H-isoquinoline-2-carboxylic acid 4,4-dimethyl-2,6-dioxo-3,4,5,6-tetrahydro-2H-[1,3′]bipyridinyl-6′-yl ester, 6,7-dimethoxy-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 3,4-dihydro-1H-isoquinoline-2-carboxylic acid 5-(7,9-dioxo-8-azaspiro[4.5]dec-8-yl)pyridin-2-yl ester, 3,4-dihydro-1H-isoquinoline-2-carboxylic acid 5-(3-thia-1-azaspiro[4.4]non- 1-en-2-ylamino)pyridin-2-yl ester, 7-Bromo-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 3,4-Dihydro-1H-isoquinoline-2-carboxylic acid 5-(2,4-dioxo-3-aza-spiro[5.5]undec-3-yl)pyridin-2-yl ester, 3,4-Dihydro-1H-isoquinoline-2-carboxylic acid 4,4-diethyl-2,6-dioxo-3,4,5,6-tetrahydro-2H-[1,3′]bipyridinyl-6′-yl ester, 3,4-Dihydro-1H-isoquinoline-2-carboxylic acid 4-ethyl-4-methyl-2,6-dioxo-3,4,5,6-tetrahydro-2H-[1,3′]bipyridinyl-6′-yl ester, 7-Chloro-3,4-dihydro-1H-isoquinoline-2-carboxylic acid 5-(7,9-dioxo-8-aza-spiro[4.5]dec-8-yl)pyridin-2-yl ester, 7-Fluoro-3,4-dihydro-1H-isoquinoline-2-carboxylic acid 5-(7,9-dioxo-8-aza-spiro[4.5]dec-8-yl)pyridin-2-yl ester, 6-Chloro-3,4-dihydro-1H-isoquinoline-2-carboxylic acid 5-(7,9-dioxo-8-aza-spiro[4.5]dec-8-yl)pyridin-2-yl ester, 6-Fluoro-3,4-dihydro-1H-isoquinoline-2-carboxylic acid 5-(7,9-dioxo-8-aza-spiro[4.5]dec-8-yl)pyridin-2-yl ester, 7-Chloro-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 7-Fluoro-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 6-Chloro-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 6-Fluoro-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 3,4-Dihydro-1H-isoquinoline-2-carboxylic acid 5-(4,4-dimethyl-4,5-dihydrothiazol-2-yl-amino)pyridin-2-yl ester, 3,4-Dihydro-1H-isoquinoline-2-carboxylic acid 5-(4,4-diethyl-4,5-dihydrothiazol-2-yl-amino)pyridin-2-yl ester, 3,4-Dihydro-1H-isoquinoline-2-carboxylic acid 5-(4-ethyl-4-methyl-4,5-dihydrothiazol-2-ylamino)pyridin-2-yl ester, 3,4-Dihydro-1H-isoquinoline-2-carboxylic acid 5-(4-methyl-2,6-dioxopiperazin-1-yl)-pyridin-2-yl ester, 3,4-Dihydro-1H-isoquinoline-2-carboxylic acid 5-(4-ethyl-2,6-dioxopiperazin-1-yl)-pyridin-2-yl ester, 3,4-Dihydro-1H-isoquinoline-2-carboxylic acid 5-(4-isobutyl-2,6-dioxopiperazin-1-yl)-pyridin-2-yl ester, 3,4-Dihydro-1H-isoquinoline-2-carboxylic acid 4,4-dimethyl-2-oxo-3,4,5,6-tetrahydro-2H-[1,3′]bipyridinyl-6′-yl ester, 7-Chloro-3,4-dihydro-1H-isoquinoline-2-carboxylic acid 4,4-dimethyl-2-oxo-3,4,5,6-tetrahydro-2H-[1,3′]bipyridinyl-6′-yl ester, 7-Fluoro-3,4-dihydro-1H-isoquinoline-2-carboxylic acid 4,4-dimethyl-2-oxo-3,4,5,6-tetrahydro-2H-[1,3′]bipyridinyl-6′-yl ester, 6-Chloro-3,4-dihydro-1H-isoquinoline-2-carboxylic acid 4,4-dimethyl-2-oxo-3,4,5,6-tetrahydro-2H-[1,3′]bipyridinyl-6′-yl ester, 6-Fluoro-3,4-dihydro-1H-isoquinoline-2-carboxylic acid 4,4-dimethyl-2-oxo-3,4,5,6-tetrahydro-2H-[1,3′]bipyridinyl-6′-yl ester, 6,7-Dimethoxy-3,4-dihydro-1H-isoquinoline-2-carboxylic acid 4,4-dimethyl-2-oxo-3,4,5,6-tetrahydro-2H-[1,3′]bipyridinyl-6′-yl ester, 7-Methoxy-3,4-dihydro-1H-isoquinoline-2-carboxylic acid 4,4-dimethyl-2-oxo-3,4,5,6-tetrahydro-2H-[1,3′]bipyridinyl-6′-yl ester, 6-Methoxy-3,4-dihydro-1H-isoquinoline-2-carboxylic acid 4,4-dimethyl-2-oxo-3,4,5,6-tetrahydro-2H-[1,3′]bipyridinyl-6′-yl ester, 3,4-Dihydro-1H-isoquinoline-2-carboxylic acid 4-isopropyl-2,6-dioxo-3,4,5,6-tetrahydro-2H-[1,3′]bipyridinyl-6′-yl ester, 7-Chloro-3,4-dihydro-1H-isoquinoline-2-carboxylic acid 4-isopropyl-2,6-dioxo-3,4,5,6-tetrahydro-2H-[1,3′]bipyridinyl-6′-yl ester, 7-Fluoro-3,4-dihydro-1H-isoquinoline-2-carboxylic acid 4-isopropyl-2,6-dioxo-3,4,5,6-tetrahydro-2H-[1,3′]bipyridinyl-6′-yl ester, 6-Chloro-3,4-dihydro-1H-isoquinoline-2-carboxylic acid 4-isopropyl-2,6-dioxo-3,4,5,6-tetrahydro-2H-[1,3′]bipyridinyl-6′-yl ester, 6-Fluoro-3,4-dihydro-1H-isoquinoline-2-carboxylic acid 4-isopropyl-2,6-dioxo-3,4,5,6-tetrahydro-2H-[1,3′]bipyridinyl-6′-yl ester, 6,7-Dimethoxy-3,4-dihydro-1H-isoquinoline-2-carboxylic acid 4-isopropyl-2,6-dioxo-3,4,5,6-tetrahydro-2H-[1,3′]bipyridinyl-6′-yl ester, 7-Methoxy-3,4-dihydro-1H-isoquinoline-2-carboxylic acid 4-isopropyl-2,6-dioxo-3,4,5,6-tetrahydro-2H-[1,3′]bipyridinyl-6′-yl ester, 6-Methoxy-3,4-dihydro-1H-isoquinoline-2-carboxylic acid 4-isopropyl-2,6-dioxo-3,4,5,6-tetrahydro-2H-[1,3′]bipyridinyl-6′-yl ester, 3,4-Dihydro-1H-isoquinoline-2-carboxylic acid 3,3-dimethyl-2,6-dioxo-3,4,5,6-tetrahydro-2H-[1,3′]bipyridinyl-6′-yl ester, 7-Chloro-3,4-dihydro-1H-isoquinoline-2-carboxylic acid 3,3-dimethyl-2,6-dioxo-3,4,5,6-tetrahydro-2H-[1,3′]bipyridinyl-6′-yl ester, 7-Fluoro-3,4-dihydro-1H-isoquinoline-2-carboxylic acid 3,3-dimethyl-2,6-dioxo-3,4,5,6-tetrahydro-2H-[1,3′]bipyridinyl-6′-yl ester, 6-Chloro-3,4-dihydro-1H-isoquinoline-2-carboxylic acid 3,3-dimethyl-2,6-dioxo-3,4,5,6-tetrahydro-2H-[1,3′]bipyridinyl-6′-yl ester, 6-Fluoro-3,4-dihydro-1H-isoquinoline-2-carboxylic acid 3,3-dimethyl-2,6-dioxo-3,4,5,6-tetrahydro-2H-[1,3′]bipyridinyl-6′-yl ester, 6,7-Dimethoxy-3,4-dihydro-1H-isoquinoline-2-carboxylic acid 3,3-dimethyl-2,6-dioxo-3,4,5,6-tetrahydro-2H-[1,3′]bipyridinyl-6′-yl ester, 7-Methoxy-3,4-dihydro-1H-isoquinoline-2-carboxylic acid 3,3-dimethyl-2,6-dioxo-3,4,5,6-tetrahydro-2H-[1,3′]bipyridinyl-6′-yl ester, and 6-Methoxy-3,4-dihydro-1H-isoquinoline-2-carboxylic acid 3,3-dimethyl-2,6-dioxo-3,4,5,6-tetrahydro-2H-[1,3′]bipyridinyl-6′-yl ester, or a pharmaceutically acceptable salt thereof.

32. A compound according to claim 31 which is selected from the group consisting of:
3,4-dihydro-1H-isoquinoline-2-carboxylic acid 4,4-dimethyl-2,6-dioxo-3,4,5,6-tetrahydro-2H-[1,3′]bipyridinyl-6′-yl ester, 6,7-dimethoxy-3,4-dihydro-1H-isoquinoline-2-carboxylic acid 4,4-dimethyl-2,6-dioxo-3,4,5,6-tetrahydro-2H-[1,3′]bipyridinyl-6′-yl ester, 3,4-dihydro-1H-isoquinoline-2-carboxylic acid 5-(7,9-dioxo-8-azaspiro[4.5]dec-8-yl)pyridin-2-yl ester, and 3,4-dihydro-1H-isoquinoline-2-carboxylic acid 5-(3-thia- 1-azaspiro[4.4]non- 1-en-2-ylamino)pyridin-2-yl ester, or a pharmaceutically acceptable salt thereof.

33. A pharmaceutical composition comprising a compound according to claim 1 together with a pharmaceutically acceptable carrier or diluent.

34. A pharmaceutical composition according to claim 33 in unit dosage form, comprising from about 0.05 to about 2000 mg, from about 0.1 to about 500 mg or from about 1.0 to about 100 mg of the compound or pharmaceutically acceptable salt thereof.

35. A method of treating a disorder wherein modulation of the activity of hormone-sensitive lipase is desired, comprising administering to a subject in need thereof a therapeutically effective amount of a compound according to claim 1, or a pharmaceutically acceptable salt thereof.

36. A method of treating a disorder wherein lowering of the activity of hormone-sensitive lipase is desired, comprising administering to a subject in need thereof a therapeutically effective amount of a compound according to claim 1, or a pharmaceutically acceptable salt thereof.

37. The method according to claim 35, wherein the disorder is selected from insulin resistance, type 1 diabetes, type 2 diabetes, metabolic syndrome X, impaired glucose tolerance, hyperglycemia, dyslipidemia, obesity, atheroschlerosis, hypertension, abnormalities of lipoprotein metabolism, and combinations thereof.

38. The method according to claim 36, wherein the disorder is selected from insulin resistance, type 1 diabetes, type 2 diabetes, metabolic syndrome X, impaired glucose tolerance, hyperglycemia, dyslipidemia, obesity, atheroschlerosis, hypertension, abnormalities of lipoprotein metabolism, and combinations thereof.

39. The method according to claim 37, wherein a further antidiabetic, antiobesity, antihypertensive or appetite regulating drug is administered to the subject.

40. The method according to claim 38, wherein a further antidiabetic, antiobesity, antihypertensive or appetite regulating drug is administered to the subject.

41. The method according to claim 39, wherein metformin is administered to the subject.

42. The method according to claim 40, wherein metformin is administered to the subject.

43. The method according to claim 37, wherein the disorder is type 2 diabetes.

44. The method according to claim 38, wherein the disorder is type 2 diabetes.

45. A method for delaying the progression from non-insulin requiring type 2 diabetes to insulin requiring type 2 diabetes, comprising administering to a subject in need thereof a therapeutically effective amount of a compound according to claim 1, or a pharmaceutically acceptable salt thereof.

46. A method of inhibiting the lipolytic activity of hormone-sensitive lipase against triacylglycerols, diacylglycerols, cholesterol acyl esters or steroid acyl esters, comprising administering to a subject in need thereof a therapeutically effective amount of a compound according to claim 1, or a pharmaceutically acceptable salt thereof.

47. The pharmaceutical composition according to claim 33, wherein the pharmaceutical composition is formulated for oral administration.

48. The pharmaceutical composition according to claim 33, wherein the pharmaceutical composition is formulated for nasal, transdermal, pulmonal, or parenteral administration.
